(19) United States
(12) Patent Application Publication
(10) Pub. No.: US 2006/0241715 A1
(43) Pub. Date: Oct. 26, 2006

(54) Electrode for a pulse generator and method therefor

(75) Inventors: Jason Sprain, Shoreview, MN (US); Robert Oskar Jensen, New Brighton, MN (US)

Correspondence Address: SCHWEGMAN, LUNDBERG, WOESSNER & KLUTH, P.A., P.O. BOX 2938, MINNEAPOLIS, MN, 55402, US

(73) Assignee: Cardiac Pacemakers, Inc.

(21) Appl. No.: 11/112,022

(22) Filed: Apr. 22, 2005

(51) Int. Cl. A61N 1/375

(52) U.S. Cl. 607/37

(57) ABSTRACT

An implantable medical device includes a pulse generator having a housing with an outer casing having internal circuitry therein and a lead connector portion. An external electrode is wrapped around at least a portion of the housing or flexibly formed around a portion of the housing. The external electrode is electrically coupled with the internal circuitry. The device further includes at least one interconnect member electrically coupled with the external electrode.

### TECHNICAL FIELD

Implanted medical devices that incorporate external electrical connections, for example, implanted cardiac rhythm management devices that include a leadless electrode on the outer surface of an implanted pulse generator device.

### BACKGROUND

Implantable medical devices, such as cardiac rhythm management devices, commonly include implanted pacemakers and defibrillator units. These devices include sensing, signal processing and control circuitry, together with a power supply protectively housed in a hermetically sealed case or can in combination with one or more conductive electrical leads designed to connect to the patient's heart muscle tissue. To maintain the integrity of the components in the sealed case, provision must be made for sealed passage of electrical conductors to the exterior for connection to the leads and ultimately to the tissue of interest. This has been typically accomplished by using connector blocks and associated feedthrough conductors located external to the implanted pulse generator housing or case which, themselves, typically are placed within a sealed lead connector structure of medical grade polymer material.

Button or similar leadless or “indifferent” sensing electrodes have also been provided on the outer surface of the lead connector portion of pulse generator cases or housing, each connected by an independent wire conductor between the electrode and the feedthrough system. These external leadless pulse generator electrodes and connecting conductors can be relatively expensive to manufacture and assemble. For instance, these electrode devices have been machined (a relatively expensive process) from medical grade stainless steels or titanium and thereafter wired to a feedthrough terminal. Wire conductive connectors have also been used between feedthrough terminals and corresponding connector blocks in the pulse generator lead/connecting portion with received and connect with the other external leads.

What is needed is a medical device including an external leadless electrode and feedthrough connection system that overcomes these and other shortcomings.

### SUMMARY

An implantable medical device, such as a pulse generator, includes a housing that has a header and an outer casing having internal circuitry therein. The implantable device further includes an external electrode wrapped around at least an outer portion of the housing, and a plurality of interconnect members electrically coupled with the external electrode and the internal circuitry. The pulse generator is optionally coupled with one or more lead assemblies.

Several options are possible for the medical device, including, but not limited to, the options as follows. For instance, the external electrode, such as a thin, flexible electrode, can be wrapped around two or three sides of the device, allowing for multi sided access to the external electrode. One example of the flexible electrode is a sheet metal electrode wrapped around the housing.

In another embodiment, an implantable medical device includes a header for receiving leads therein, where the header includes an electrode mounting portion with two or more sides. The device further includes at least one electrode flexibly formed around at least a portion of the electrode mounting portion, and an interconnect electrically coupling the at least one external electrode with circuitry within the device. The electrode is optionally coupled with the housing of the device, for example, by retention features, such as, but not limited to, crimped features, snap fit features, or interference fit.

A method is further described herein that includes wrapping an external electrode around at least a portion of a housing of a pulse generator, coupling the external electrode with the housing, coupling the external electrode with at least one interconnect an internal circuitry.

Several options are possible for the method, including, but not limited to, the options as follows. For instance, the electrode can be wrapped around one or more sides of the housing, and/or the header of the pulse generator. In another option, the electrode can be coupled with the housing in a number of different ways, such as interference fit, snap fit, forming crimp(s) in the electrode, etc.

These and other embodiments, aspects, advantages, and features will be set forth in part in the description which follows, and in part will become apparent to those skilled in the art by reference to the following description and referenced drawings or by practice thereof. The aspects, advantages, and features are realized and attained by means of the instrumentalities, procedures, and combinations particularly pointed out in the appended claims and their equivalents.

### BRIEF DESCRIPTION OF THE DRAWINGS

In the drawing figures wherein like reference characters depict like parts throughout the same:

FIG. 1a illustrates a perspective view of a portion of an implantable pulse generator device constructed in accordance with at least one embodiment;

FIG. 1b illustrates an exploded perspective view of an implantable pulse generator device constructed in accordance with at least one embodiment;

FIG. 2 is a cross-sectional view through the lead connector of FIG. 1 taken along 2-2;

FIG. 3 illustrates a portion of a pulse generator device constructed in accordance with at least one embodiment;

FIG. 4 is a cross-sectional view through the lead connector of FIG. 3 taken along 4-4; and

FIG. 5a illustrates a portion of a pulse generator device constructed in accordance with at least one embodiment;

FIG. 5b illustrates a portion of a pulse generator device constructed in accordance with at least one embodiment;

FIG. 6 illustrates a top plan view of a preformed external electrode constructed in accordance with at least one embodiment;

FIG. 7a illustrates a perspective view of an external electrode constructed in accordance with at least one embodiment; and

FIG. 7b illustrates a side view of an external electrode constructed in accordance with at least one embodiment.

### DETAILED DESCRIPTION

In the following detailed description, reference is made to the accompanying drawings, which form a part hereof, and in which is shown by way of illustration specific embodiments in which the invention may be practiced. These embodiments are described in sufficient detail to enable those skilled in the art to practice the invention, and it is to be understood that other embodiments may be utilized and that structural changes may be made without departing from the spirit and scope of the present invention. Therefore, the following detailed description is not to be taken in a limiting sense, and the scope is defined by the appended claims.

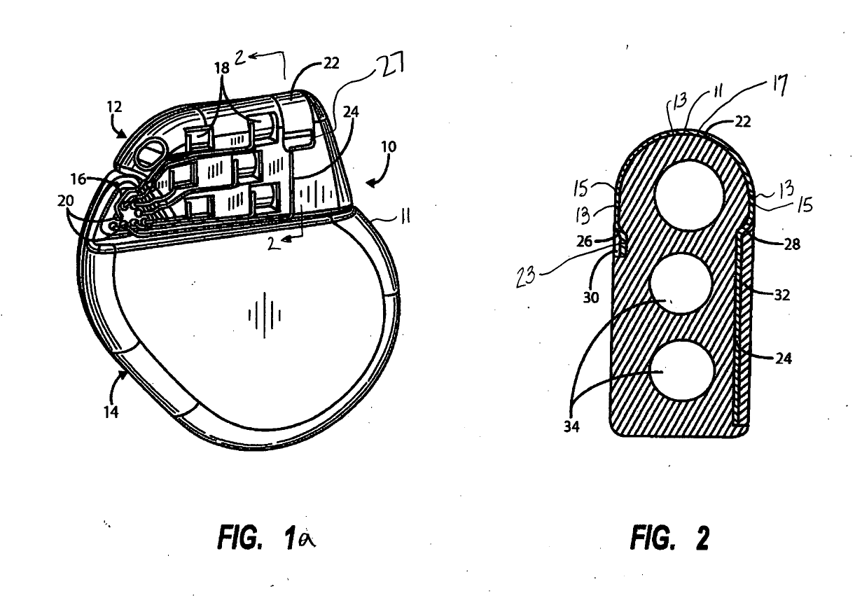

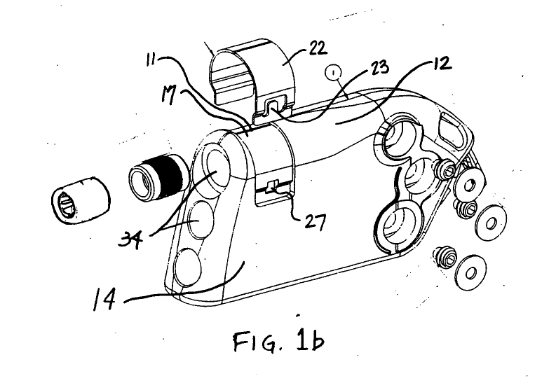

FIGS. 1a and 1b illustrate one example of an implantable medical device, for instance, an implantable pulse generator device 10, that allows for signals to be sent and/or received from tissue, for example, signals to and from a heart. The implantable pulse generator device 10 includes an outer housing 11 having an optional lead connector portion 12, and a casing 14. The housing 11 is defined in part by one or more planes 13 (FIG. 2). For example, the housing 11 includes two major planes 15 (FIG. 2). The term “plane” is not intended to be limited to entirely nor substantially flat surfaces, however, it does include the sides, top and bottom of the housing 11. The implantable pulse generator device 10 further includes at least one external electrode 22 disposed along, in one example, the lead connector portion 12.

The lead connector portion 12, or the header, allows for elongate leads to be mechanically and electrically coupled therein. The lead is, for example, an elongate structure that includes electrodes disposed therealong. Electrodes disposed along the lead can be disposed in, on, along, or near tissue to be sensed or paced, such as a heart. The lead connector portion 12 is, in one option, generally fabricated from medical grade polymer material, which includes a number of openings 34 therein. The openings 34 allow for the optional leads to be inserted and retained therein. The openings 34 contain electrical connections which allow for the lead to be electrically coupled with various circuitry within the casing 14.

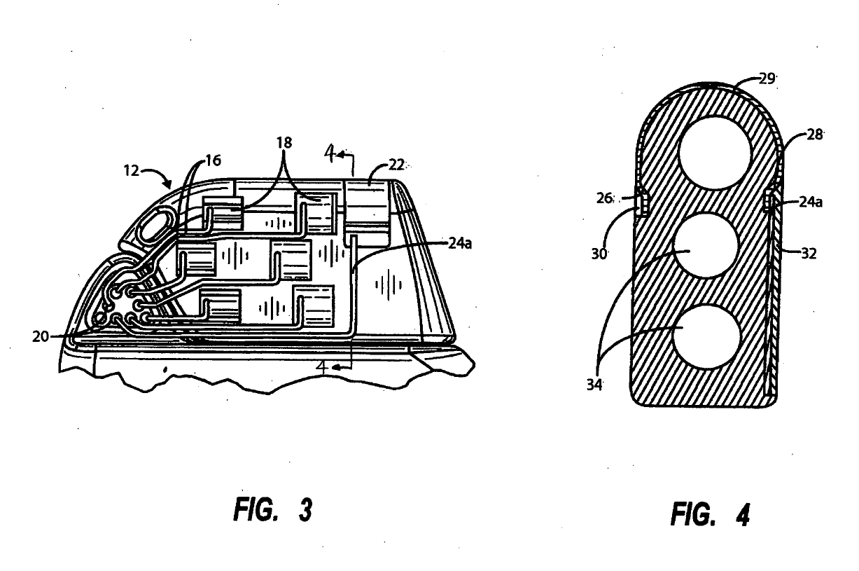

The lead connector portion 12 further includes, in one example, an electrode mounting portion 17, and at least one external electrode 22, for example, a leadless or indifferent electrode, disposed along the electrode mounting portion 17. The external electrode 22 is, in one option, a thin, readily shaped electrode 22 with corresponding interconnect 24 (FIG. 2) connecting the electrode 22 with one of the feedthrough terminals 20 (FIG. 3), as further described below. The interconnect 24 can be fabricated as an integral part of the electrode 22, as shown in FIGS. 1a or 2. This illustrative embodiment depicts the leadless or indifferent electrode and interconnect 24 as a single, continuous fabricated unitary structure. FIGS. 3 and 4 depict an alternative embodiment in which the leadless or indifferent electrode 22 is connected to a separate connecting conductor 24a.

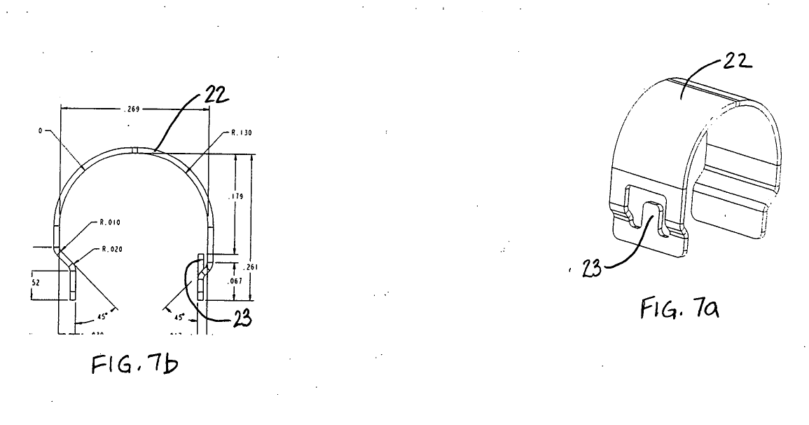

The external electrode 22 is an electrode that, in one option, is wrapped around a portion of the header. The wrapped external electrode 22 covers the portion of the header, for example, by winding, folding, or coating a substantially non-rigid electrode around a portion of the header. In one example, the external electrode 22 is a thin and/or flexible member, such as sheet metal having a thickness and/or treatment allowing it to be wrapped or alternatively flexibly formed around a portion of the header. One example is illustrated in FIGS. 7a and 7b, where the electrode 22 is formed or wrapped into a curved shape having a similar cross section to that of the outer electrode mounting portion 17 (FIG. 2), and in one example has a C shape.

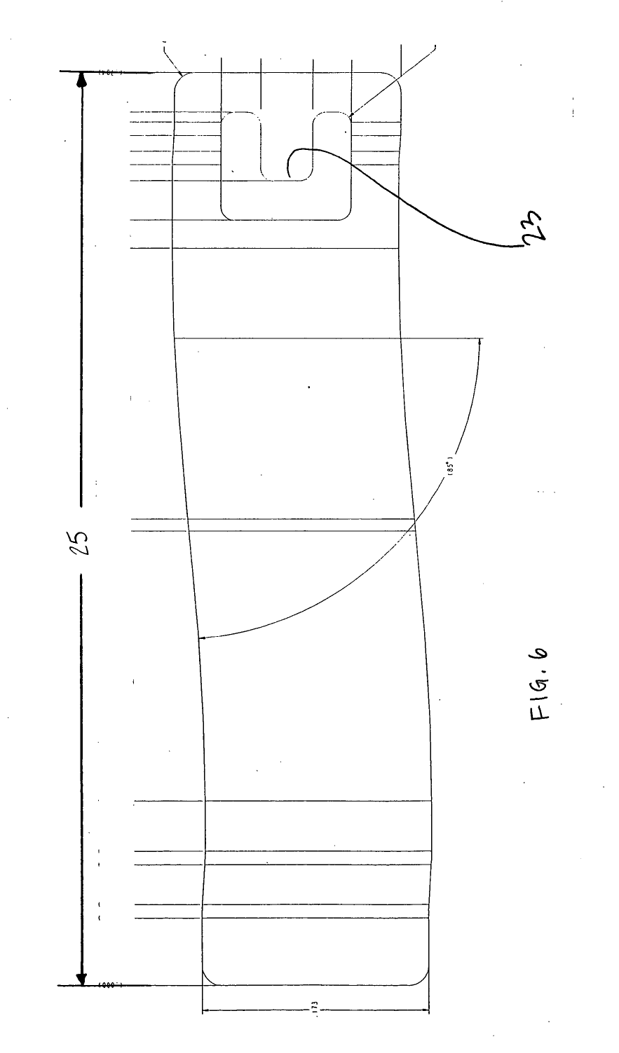

The external electrode 22, in one example, includes features that allow for the electrode to be coupled, secured, or retained with the housing 11 (FIG. 2). In one example, the shape is smaller than the shape of the outer electrode mounting portion 17, allowing for the electrode 22 to have an interference fit with the housing 11 (FIG. 2). In another example, the external electrode 22 includes a crimped member 23 (FIG. 6, 7a), that couples with a portion 27 (FIG. 1a) of the housing 11 (FIG. 1a), for example with molded indents of the housing 11. Note that the electrode 22 is crimped at 26, 28, respectively, to improve retention, in one example. In yet another option, the external electrode 22 and/or the housing 11 (FIGS. 1a and 1b) include features that allow for the external electrode 22 to be snap-fitted and/or interference fitted with the housing 11 (FIGS. 1a and 1b). Optionally, overlayers of medical grade polymer at 30, 32 assist in retaining the electrode 22 in place when the assembly is completed.

The preformed or prewrapped electrode 22, shown in FIG. 6, has a length 25, in one option, allowing for the electrode 22 to be wrapped around various portions of the housing 11. For example, the electrode 22 has a length 25 allowing the electrode 22 to be wrapped and/or formed around two or more planes 13 (FIG. 2). In another option, the electrode 22 has a shape and length to be wrapped and/or formed over, along, or adjacent two major planes 15. In yet another option, the electrode 22 has a shape and length to be wrapped and/or formed over, along, or adjacent three or more planes. After the electrode 22 is formed, it has, in at least one option, a curved shape, such as a C shape as illustrated in FIG. 7b.

The external electrode 22 is electrically isolated from the casing 14, in a number of different manners. For example, the header can be formed of various non-conductive materials including, but not limited to, polyurethane, or epoxy, and the external electrode 22 is coupled with the header. In another option, other insulative material and/or coating can be disposed between the external electrode 22 and the header and/or the casing.

As discussed above, the electrode 22 is disposed along or optionally coupled with the housing 11, which includes a casing 14, as shown in FIGS. 1a and 1b. The casing 14 is an outer casing that houses electronic circuitry therein. The casing 14 is, in one or more option, generally made of titanium or medical grade stainless steels. The circuitry contained within the housing includes circuitry for sensing, signal processing, and control circuitry, along with a power supply. The internal circuitry is electrically coupled with the external electrode, for example, via interconnects.

FIGS. 1a, 1b, 3, 5a and 5b, show part of the molded or filled medical grade polymer removed to illustrate the internal interconnects between certain components. These include a plurality of interconnection conductors as at 16 which may be ribbon conductors which connect a plurality of connector blocks, as at 18, with a plurality of corresponding external feedthrough terminals as at 20. The feedthrough terminal 20 may be in the form of protruding wire stubs or metal pads of conductive material compatible with the leads. The connector blocks 18, individual lead 16 and corresponding terminals 20 are insulated from each other, in at least one option.

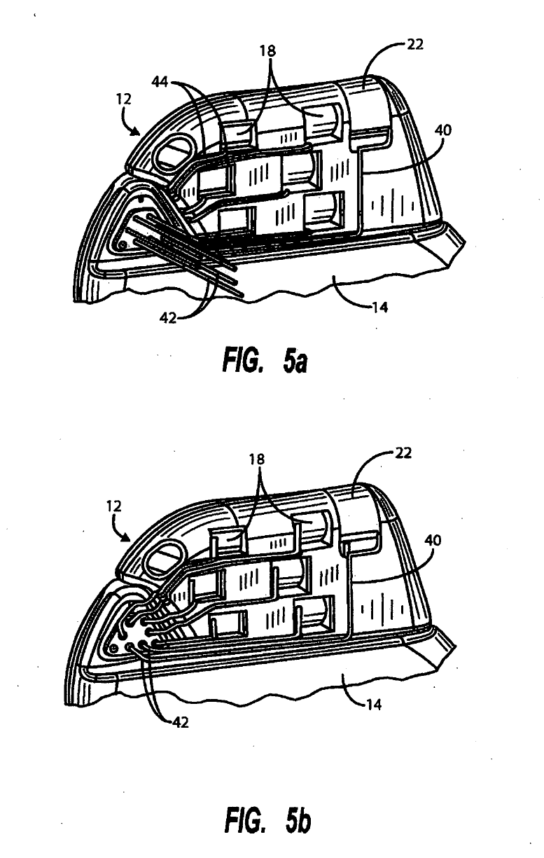

FIGS. 5a and 5b illustrate an alternative embodiment of a feed-through connection system including the leadless or indifferent electrode 22. In one example, the leadless or indifferent electrodes 22 are provided with separate leads 40, which are solid wire leads typically placed in a narrow channel provided along the upper surface of the lead connector portion 12 which may be thereafter filled with an amount of medical grade polymer, if desired.

As shown in FIGS. 5a and 5b, the plurality remaining connectors 42 are in the form of solid wires which lead to a solid wire feedthrough system. FIG. 5a depicts an array of channels as at 44 for receiving the plurality of wire connectors 42. The array is shown connected in FIG. 5b.

The conductors or interconnects 16 are in one option, in ribbon form, and are, in another option, manufactured of very thin gauge metal or foil such as a medical grade stainless steel or titanium. The external leadless electrodes are also manufactured of relatively thin gauge material. For example, the thickness for the material is from about 0.005 inches to about 0.025 inches and the width of the conductors is from about 0.020 inches to about 0.060 inches. In another example, the thickness of the material is about 0.020 inches.

The relatively thin gauge and/or flexible electrode 22 replaces a rather heavy gauge machined external button electrode, or the like. The use of the relatively thin gauge, readily shaped or wrapped metal electrodes for the leadless or indifferent electrodes eliminates the need for expensive machining which has been required for the early button-type electrodes. Furthermore, the electrode snaps on allowing for ease in manufacturability.

A method for forming an implantable device such as a pulse generator includes wrapping the external electrode 22 around at least a portion of a housing 11, such as wrapping or forming the external electrode 22 around the header. The wrapping or forming process can include the various embodiments discussed above. For example, although not so limited, the thin flexible electrode is wrapped around two or more sides of the housing, such as the header. In another option, the thin, flexible electrode is wrapped around three or more sides of the housing. For instances, the flexible electrode would be wrapped over at least a portion of a first major side, a top portion, and at least a portion of a second major side of the housing.

The external electrode 22 is also coupled with the housing 11, for example, by a snap fit and/or coupling at least one crimp of the external electrode with the housing 11. The external electrode 22 is electrically coupled with at least one interconnect, which is electrically coupled with internal circuitry of the device.

Once electrical connections are made to all of the corresponding feedthrough terminals, medical grade polymer is over-molded or backfilled in a layer which covers and preserves the connections while completing the desired outer shape of the structure, and assists with sealing the interconnect members. In this manner, the flexible interconnects are imbedded in the overlayer of medical grade polymer which is typically silicone or a medical grade polyurethane. The device is implanted and optionally coupled with a lead, for example, a lead that is implanted within a heart of a patient.

It is to be understood that the above description is intended to be illustrative, and not restrictive. Although the use of the implantable device has been described for use with a lead in, for example, a cardiac stimulation system, the implantable device could as well be applied to other types of body stimulating systems. Many other embodiments will be apparent to those of skill in the art upon reviewing the above description. The scope should, therefore, be determined with reference to the appended claims, along with the full scope of equivalents to which such claims are entitled.

What is claimed is:

1. An implantable medical device comprising:
a housing including a header and an outer casing having internal circuitry therein;
an external electrode wrapped around at least an outer portion of the housing; and
one or more interconnect members electrically coupled with the external electrode and the internal circuitry.

2. The implantable medical device as recited in claim 1, wherein the external electrode is wrapped around at least a portion of the header.

3. The implantable medical device as recited in claim 1, wherein the outer portion of the housing is defined by two or more major planes, and the external electrode is wrapped along the two or more planes.

4. The implantable medical device as recited in claim 1, wherein the outer portion of the housing is defined by three or more planes, and the external electrode is wrapped along the three or more planes.

5. The implantable medical device as recited in claim 1, wherein the internal interconnect members include ribbon conductors.

6. The implantable medical device as recited in claim 1, wherein the header includes lead connector openings adapted to receive a lead therein.

7. The implantable medical device as recited in claim 1, wherein the external electrode is integral with at least one interconnect.

8. The implantable medical device as recited in claim 1, wherein the external electrode is a sheet metal formed electrode.

9. An implantable medical device comprising:
an outer housing having a header including an electrode mounting portion with two or more sides, the outer housing further including a casing;
at least one electrode flexibly formed around at least a portion of the electrode mounting portion; and
an interconnect electrically coupling the at least one external electrode with circuitry within the outer housing.

10. The implantable medical device as recited in claim 9, wherein the electrode has a C-shaped cross section.

11. The implantable medical device as recited in claim 9, wherein the external electrode includes at least one electrode retention feature.

12. The implantable medical device as recited in claim 11, wherein the retention member comprises a crimped portion of the electrode.

13. The implantable medical device as recited in claim 9, wherein the electrode is coupled with a portion of the housing with an interference fit.

14. The implantable medical device as recited in claim 9, wherein the electrode is electrically isolated from the casing.

15. A method comprising:
wrapping an external electrode around at least a portion of an outer housing of a pulse generator;
mechanically coupling the external electrode with the housing; and
coupling the external electrode with at least one interconnect and internal circuitry.

16. The method as recited in claim 15, further comprising wrapping the electrode around two or more sides of the housing.

17. The method as recited in claim 15, wherein wrapping includes wrapping around a header of the pulse generator.

18. The method as recited in claim 17, further comprising insulating between the external electrode and a casing of the pulse generator.

19. The method as recited in claim 15, further comprising coupling a lead with the outer case.

20. The method as recited in claim 15, wherein coupling the external electrode includes coupling a crimped portion of the external electrode with the housing.

21. The method as recited in claim 15, wherein coupling the external electrode includes snap-fittedly coupling the external electrode with the housing.
